(19) United States
(12) Patent Application Publication
(10) Pub. No.: US 2026/0224875 A1
(43) Pub. Date: Aug. 6, 2026

(54) METHOD TO ENHANCE TRANSPORT ACROSS THE BLOOD-BRAIN BARRIERVIA VASODILATION TRIGGERED BY VIBROACOUSTIC THERAPY AND/OROTHER STACKED SENSORY INPUTS ALLOWING FOR SYNERGISTICBENEFITS WITH PHARMACOLOGICAL TREATMENTS FOR NEUROLOGICALAND CIRCULATORY DISEASES

(71) Applicant: Kuranda Services LLC, Forest Lakes, AZ (US)

(72) Inventors: Shawn Leo Gallagher, Dillon, CO (US); Delton Leon Hyatt, Phoenix, AZ (US)

(73) Assignee: Kuranda Services LLC, Forest Lakes, AZ (US)

(21) Appl. No.: 19/181,976

(22) Filed: Apr. 17, 2025

Related U.S. Application Data
(63) Continuation of application No. 19/042,761, filed on Jan. 31, 2025.

(51) Int. Cl. A61M 37/00; A61H 1/00; A61K 31/13; A61K 31/137; A61K 31/198; A61K 31/381; A61K 31/451; A61K 31/4525; A61K 31/4745; A61K 39/395; A61K 41/00; A61M 21/00; A61M 21/02

(52) U.S. Cl. CPC A61M 37/0092; A61H 1/005; A61K 31/13; A61K 31/137; A61K 31/198; A61K 31/381; A61K 31/451; A61K 31/4525; A61K 31/4745; A61K 39/3955; A61K 41/0047; A61M 21/02; A61H 2201/105; A61H 2201/5005; A61M 2021/0027; A61M 2021/0077; A61M 2210/0693

(57) ABSTRACT

Disclosed are methods and systems for improving treatment efficacy of circulatory and neurological disorders. Stacked sensory inputs are used to stimulate vascular endothelium to release nitric oxide, counteract endothelial dysfunction, and promote vasodilation. Combining vibration with administration of one or more therapeutic modalities provides for synergistic effects on treatment of circulatory and neurological disorders.

### FIELD OF THE INVENTION

The present invention relates to systems, apparatus, and methods for treating clinical conditions such as circulatory and neurological disorders. In particular, the present invention relates to synergistic combination of multiple, stacked sensory inputs with other therapeutic modalities to more effectively treat circulatory and neurological disorders. Furthermore, the present invention relates to synergistic combination of vibro acoustic therapy (VAT) and/or whole body vibration (WBV) with other therapeutic modalities, such as specific music delivery or pharmacological treatments, to treat neurological disorders.

### BACKGROUND OF THE INVENTION

Our nervous system consists of the central nervous system (CNS) and peripheral nervous systems. The CNS is made up of the brain and spinal cord, while the peripheral nervous system includes all other nerves. There are hundreds of neurological disorders, defined as disorders that affect the brain as well as the nerves throughout the spinal cord and the rest of the human body. Common neurological disorders include neurodegenerative conditions such as various types of dementia and brain tumors.

Neurodegenerative diseases are age-dependent disorders with very different pathophysiologies. As the world population is increasingly becoming older, these diseases are becoming more prevalent. The World Health Organization (WHO) has predicted that neurodegenerative diseases affecting motor function will become the second-most prevalent cause of death in the next 20 years.

Alzheimer's disease is the most common neurodegenerative disease, accounting for 80% of dementia cases in elderly people. In the United States, as many as 6.9 million people may have Alzheimer's disease, according to a report from the Alzheimer's Disease Association in 2024. Its symptoms include loss of memory, language deterioration, and visuospatial deficits.

Although the cause for Alzheimer's disease is not yet fully elucidated, it is widely accepted that Alzheimer's disease has to do with accumulation of amyloid plaques containing amyloid-β peptides and neurofibrillary tangles made of tau protein, which leads to neuronal and synaptic loss. The buildup may be due to overproduction, reduced clearance, or both.

The brain's waste removal system, or glymphatic system, is impaired in Alzheimer's disease and other conditions that involve toxic waste buildup. The glymphatic system uses cerebrospinal fluid (CSF) to clear away toxic waste. As people age, the glymphatic system's effectiveness decreases.

Sleep disturbances have been linked to dementia and cognitive decline in older adults, although the causal relationship between sleep and Alzheimer's disease is not clear. Regardless, various studies have shown that sleep, especially deep sleep, helps the brain clear out waste products, including amyloid buildups. On the other hand, waste buildup may negatively affect sleep quality and worsen progression of Alzheimer's disease or other neurodegenerative disorders.

Parkinson's disease is the second most common neurodegenerative disease. It is most known for its motor control symptoms despite several non-motor related symptoms, such as cognitive impairment, sleep disorders, and depression.

Currently, no neurodegenerative disease is curable, and the treatments available only manage symptoms or halt/slow the progression of the disease.

One problem with introducing therapeutic agents into the brain is the blood-brain barrier (BBB). The BBB lines the capillaries of the brain and is comprised of endothelial cells connected by tight junctions. It allows for selective exchange of substances between the systemic circulation and the extracellular fluid compartment of the brain. Only small lipophilic molecules can pass the BBB by free diffusion. Furthermore, the BBB contains active transporter proteins, but only molecules that match one of these transporters (e.g., glucose) can be actively transferred across the endothelial cells. The BBB restricts the entry from the blood into the brain of pathogens, hydrophilic molecules, and macromolecules; it also regulates the export of toxic molecules from the brain (likely overlapping with glymphatic clearance). A need therefore exists for systems and methods to improve transfer of substances across the BBB, including both entry of therapeutic agents into the brain and export of waste from the brain, in order to enhance treatment efficacy or slow progression of neurological disorders.

There are two other sites in the CNS that form a barrier between the blood and CSF: the arachnoid epithelium forming the middle layer of the meninges and the choroid plexus epithelium. At each site, tight junctions reduce the permeability of the intercellular adhesion areas.

The circulatory system, also called cardiovascular system, keeps blood moving in a body. Circulatory diseases affect the heart or blood vessels and make it harder for blood to flow throughout the body. Some symptoms are minor, while others are life-threatening. Well-known circulatory diseases include heart attack, heart failure, high blood pressure, etc.

It has been found that particular modes of sound or music delivered to an observer can produce a variety of effects, called vibroacoustic effects, in the observer's brain. Such delivery can include vibrations delivered to the observer's body through an interface, for example without limitation, through a floor or through furniture disposed on the floor, and can be beneficial for the mental health of the observer. In particular, low-frequency vibration, typically between about 20 Hz and about 250 Hz and having sufficient amplitude to generate significant tactile appreciation, has been shown to improve or slow the progression of Parkinson's and other neurodegenerative diseases.

U.S. Pat. No. 12,143,765 discloses a platform for delivery to a user of vibrations with controlled frequencies and amplitudes. The platform is supported to freely vibrate without dampening of the applied vibrations. Furthermore, the platform is capable of supporting a user so that rhythmic body movements and stretching of the user could be incorporated into the vibroacoustic effects to produce enhanced beneficial outcomes for the user.

Low-frequency vibration, such as vibro acoustic therapy (VAT) or whole body vibration (WBV), in the right environment can improve many health and performance related issues. Vagal Integration, Balanced Neuromodulation, Audio and Vibro Acoustics (VIBNAVA) has demonstrated improvement in 1) sleep quality; 2) neuromodulation; 3) stimulation of the parasympathetic nervous system; 4) vasodilation and 5) a reduction in anxiety and stress. In addition, these five key outcomes of VIBNAVA may be durable post-treatment and, when combined contemporaneously with a therapeutic agent (e.g. an anti-amyloid protein therapy for Alzheimer's disease) will generate a synergistic effect that improves outcomes significantly beyond the additive result which would be observed if these therapeutic interventions are supplied separately.

One important benefit of low-frequency vibration is vascular dilation (vasodilation). By positively stimulating the ventral vagal nerve, such as with VAT or WBV, a subject is led to be calm and relaxed, with the perception of a safe and secure environment. Next, a high “right brain centric” workload, such as RBSDS (Right Brain Sound Delivery System) music, is introduced to get the subject into a meditative state. Then the endothelial cells of the subject are stimulated by shear forces resulting from applied vibration to release nitric oxide and create vasodilation along with the intended neurological regulation effects.

It is known in the art that one of the prominent roles of nitric oxide is to relax blood vessels, which improves blood flow and oxygen supply as well as lowers blood pressure. Relatedly, nitric oxide can improve brain function by providing adequate blood flow to supply oxygen and nutrients to the brain and removing cellular waste. Nitric oxide has other health benefits: it can also improve exercise performance, wound healing, and reproduction.

Deficiency of nitric oxide is common, especially in advanced age. This is because the body produces less nitric oxide as one ages. Low levels of nitric oxide are associated with high blood pressure, depression, poor vision, fatigue, and memory loss.

The body naturally produces nitric oxide as a free radical. It is the end product of a conversion process of dietary nitrates and vitamin C. In addition to increased release upon low-frequency vibration, nitric oxide levels in the body can also be increased by certain foods, such as beets, leafy greens, citrus fruits, nuts, poultry, garlic, and cocoa.

Endothelial dysfunction is a term that covers diminished production/availability of nitric oxide and an imbalance in the relative contribution of endothelium-derived relaxing and constricting factors. Endothelial dysfunction is a hallmark of impairment of vasculature and plays a pivotal role in the development of many types of human diseases, such as cardiovascular diseases (e.g., coronary artery disease), metabolic diseases (e.g., diabetes), cerebrovascular diseases (e.g., stroke), and neurological disorders (e.g., dementia). Cardiovascular exercise, sex, and heat can improve function of endothelial cells.

Therefore, stacked sensory inputs, for example via vibrational therapy, can be used to stimulate vascular endothelial cells, increase nitric oxide release, induce vasodilation, alleviate endothelial dysfunction, and synergistically improve treatment efficacy of circulatory and neurological diseases and disorders.

### BRIEF SUMMARY OF THE INVENTION

Provided herein are methods, apparatus, and systems for synergistically increasing the treatment effect of a therapeutic modality. Specifically, provided herein are systems, apparatus, and methods for increasing delivery of at least one therapeutic agent to the target site(s) and/or enhancing removal of at least one toxic agent in a patient and thereby increasing clinical efficacy of the therapeutic modality/agent.

Provided are methods for treating at least one neurological disorder in a patient, and the method involves administration to the patient within a period of time a combination of low-frequency vibration and at least one other therapeutic modality. The combination treatment is expected to achieve more than an additive, that is synergistic, effect compared with the sum of the effect of each treatment when administered alone.

Also provided are apparatus for delivering to a patient vibration of an appropriate frequency, amplitude, and duration, wherein the vibration promotes vasodilation and increases bi-directional transfer across the patient's blood-brain barrier while lowering blood pressure.

Further provided are combinatorial systems for treating at least one neurological disorder in a patient, and the system includes an apparatus for delivering to a patient vibration of an appropriate frequency, amplitude, and duration as well as at least one other therapeutic modality for treating the neurological disorder. The vibration delivered by the apparatus promotes vasodilation, increases bi-directional transfer across the patient's blood-brain barrier, reduces stress, and enhances treatment effect of each therapeutic modality.

### BRIEF DESCRIPTION OF DRAWINGS

FIG. 1 is a schematic bottom view of a sheet of material in accordance with an exemplary embodiment of the invention.

FIG. 2 is a schematic view of a system for exciting a platform in accordance with an embodiment of the invention.

FIG. 3 is a schematic top plan view of a system for exciting a platform according to another embodiment of the invention.

### DETAILED DESCRIPTION OF INVENTION

The following detailed embodiments presented herein are for illustrative purposes. That is, these detailed embodiments are intended to be exemplary of the present invention for the purposes of providing and aiding a person skilled in the pertinent art to readily understand how to make and use the present invention. While certain shapes and materials are used in some embodiments, they are by no means an intention of restriction.

Provided are methods for treating at least one clinical condition, and the method involves administration to a patient a combination of low-frequency vibration and at least one other therapeutic modality, such as a therapeutic agent, within a period of time. The one or more other therapeutic modalities are not vibrational. The combination treatment is expected to achieve more than additive, that is synergistic, effects compared with each treatment when administered alone. In a preferred embodiment, the clinical condition is a neurological disorder.

Vibration and another therapeutic modality do not have to be administered simultaneously. In a preferred embodiment, the patient sleeps after the vibration therapy. In another preferred embodiment, the patient takes the vibration session and the therapeutic agent both before sleep for the night.

In an exemplary embodiment, administration of vibration therapy is more frequent than that of administration of another therapeutic modality. In another exemplary embodiment, administration of vibration therapy is as frequent as that of administration of the therapeutic modality. In a further exemplary embodiment, administration of vibration therapy is less frequent than that of administration of the therapeutic modality.

In an exemplary embodiment, vibration is generated and maintained by mechanical means. In another exemplary embodiment, vibration is generated and maintained acoustically. In a further exemplary embodiment, vibration is generated and maintained via any combination of acoustic vibrational energy and mechanical vibrational energy. In a further exemplary embodiment, vibration is generated and maintained via alternating cycles of acoustic vibrational energy and mechanical vibrational energy. In a preferred embodiment, the platform is excited with acoustic vibrational energy.

Vibration therapy can be administered according to a protocol. Vibration should be of a frequency and amplitude sufficient to generate significant tactile appreciation in the patient. In a preferred embodiment, the vibration therapy places a user into a ventral vagal state. In another preferred embodiment, the vibration therapy increases natural nitric oxide release in a patient. In a further preferred embodiment, the vibration therapy increases bi-directional transfer of substances across the BBB.

The duration of the vibration therapy is 20 minutes or less, 40 minutes or less, 60 minutes or less, or 120 minutes or less. A vibration session can be comprised of multiple sub-sessions or pulses of vibration and rest periods. A patient may have up to seven sessions each week.

In an exemplary embodiment, the frequency of vibration is between about 20 Hz and 250 Hz. In another exemplary embodiment, the frequency of vibration is between about 20 Hz and 120 Hz. In a preferred embodiment, the frequency of vibration is 40 Hz.

In an exemplary embodiment, the maximum amplitude of vibration is from about 0.001 mm to about 25 mm.

In an exemplary embodiment, vibration is a sinusoidal vibration. In another exemplary embodiment, vibration is not a sinusoidal vibration.

In an exemplary embodiment, vibration is horizontal. In another exemplary embodiment, vibration is vertical. In a further exemplary embodiment, vibration is both horizontal and vertical. In a further exemplary embodiment, vibration alternates between horizontal vibration and vertical vibration.

There is no limitation as to how the patient receives vibration. Vibration can be administered to the patient globally or focally. The patient may be in contact with an apparatus capable of vibrating, and vibrational energy is applied to the apparatus so that the apparatus vibrates and delivers vibration to the patient in contact. The apparatus can be of different sizes, shapes, weights, or other configurations. The apparatus can be a platform, a piece of furniture, a wearable, a personal item, or a 3D object.

The patient may receive vibration via contact with an apparatus capable of delivering vibration to the patient, such as a vibrating platform. In an exemplary embodiment, the patient is in direct contact with a vibrating platform. The patient can be in contact with the vibration platform in various ways. For example, the patient can be standing, moving, dancing, sitting, resting, lying, stretching on the vibrating platform, or otherwise in contact with the platform. In another exemplary embodiment, the patient is subject to vibration via indirect contact with the vibrating platform, such as through a piece of furniture not vibrating by itself. The patient can be in partial contact or full contact with the vibrating platform. In a preferred embodiment, the patient is in full contact with the vibrating platform, such as lying on a vibrating floor.

There is no restriction as to the kind of other therapeutic modality to be used in combination with vibration. The other therapeutic modality may be, but not limited to, a therapeutic agent, such as a drug. The other therapeutic modality may be based on light, such as infra-red light or near-infrared light. The other therapeutic modality employed with vibration is used to prevent, ameliorate, treat, or cure any aspect of a clinical condition at any stage of the clinical condition, regardless of the underlying mechanism. In a preferred embodiment, the therapeutic modality is a therapeutic agent used to prevent, ameliorate, treat, or cure a neurological disorder.

In an exemplary embodiment, at least one therapeutic modality other than vibration is administered to the patient during the vibration session. In another exemplary embodiment, at least one other therapeutic modality is administered to the patient after the vibration session. In a preferred embodiment, the patient takes the other therapeutic modality within 24 hours after the vibration session. In a further exemplary embodiment, after at least one other therapeutic modality is administered to the patient, the patient is subject to vibration. In a preferred embodiment, the patient is subject to the vibration session within 24 hours after taking the therapeutic modality.

The therapeutic modality can be fast-or slow-acting. In an exemplary embodiment, the therapeutic agent is a slow-release drug. In another exemplary embodiment, the therapeutic agent is an immediate-release drug.

The therapeutic agent can be a small or large molecule. The therapeutic agent can be a compound, peptide, protein, lipid, carbohydrate, cell, or any combination thereof. The therapeutic agent can be delivered on a carrier, such as a nanocarrier like a liposome.

The therapeutic modality can target any part of a body. In an exemplary embodiment, the therapeutic agent targets the CNS. In a preferred embodiment, the therapeutic agent targets the brain.

More than one other therapeutic modality can be used in combination with vibration. In an exemplary embodiment, one therapeutic modality is administered in combination with vibration. In another exemplary embodiment, more than one therapeutic modalities are administered in combination with vibration.

The therapeutic modality can be administered to a patient via various routes, such as but not limited to oral, inhalational, dermal, and parenteral routes. The therapeutic modality can be administered via more than one route. In an exemplary embodiment, the therapeutic agent is administered enterally. In another exemplary embodiment, the therapeutic agent is administered epidurally. In a further exemplary embodiment, the therapeutic agent is administered through intravenous injection. In a further exemplary embodiment, the therapeutic agent is administered through intraarterial injection. In a further exemplary embodiment, the therapeutic agent is administered through intranasal delivery, with or without photostimulation. Photostimulation is, for example without limitation, near-infrared photostimulation. In a preferred embodiment, the therapeutic agent is administered via subcutaneous injection.

In an exemplary embodiment, the neurological disorder is a neurodegenerative disease, such as Alzheimer's Disease, vascular dementia, frontotemporal dementia, Lewy body dementia, another dementia, Parkinson's Disease, chronic traumatic encephalopathy, Huntington's disease, Amyotrophic lateral sclerosis, or multiple sclerosis. In another exemplary embodiment, the neurological disorder is a mental illness, such as PTSD, Schizophrenia, depression, or anxiety. In a further exemplary embodiment, the neurological disorder is epilepsy, stroke, or seizure. In a further exemplary embodiment, the neurological disorder is a type of brain tumor such as glioblastoma. In a further exemplary embodiment, more than one neurological disorder in the same patient is targeted for treatment by the present invention.

In an exemplary embodiment, the circulatory disease is atherosclerosis. In another exemplary embodiment, the circulatory disease is high blood pressure or hypertension. In a further exemplary embodiment, the circulatory disease is coronary artery disease. In a further exemplary embodiment, the circulatory disease is stroke.

In an exemplary embodiment, the therapeutic agent is an anti-amyloid and/or anti-tau drug for Alzheimer's disease, for example without limitation, aducanumab, donanemab, or lecanemab. In another exemplary embodiment, the therapeutic agent is medication for Parkinson's disease, for example without limitation, carbidopa, levodopa, or rotigotine. In a further exemplary embodiment, the therapeutic agent is a drug used in the treatment of Huntington's disease, such as haloperidol, tetrabenazine, or amantadine. In a further exemplary embodiment, the therapeutic agent is a medicine used to treat dementia. In a further exemplary embodiment, the therapeutic agent is used to treat sclerosis. In a further exemplary embodiment, the therapeutic agent is a drug used in the treatment of PTSD, such as paroxetine or venlafaxine. In a further exemplary embodiment, the therapeutic agent is a drug used in treatment of a brain tumor. In a further exemplary embodiment, the therapeutic agent is a combination of drugs used clinically to treat a neurological disorder.

In an exemplary embodiment, one or more other therapeutic modalities are used to treat one neurological disorder in a patient. In another exemplary embodiment, one or more other therapeutic modalities are used to treat one or more neurological disorders in the same patient.

In an exemplary embodiment, music is played during vibration therapy. The music is preferably instrumental music, meaning music that includes no vocals. The music is preferably filtered to remove predetermined frequencies, such as frequencies that place a listener into a more agitated state. These can include high-frequency sounds and other frequencies that stimulate the sympathetic nervous system, which can lead to an increased heart rate, blood vessel constriction, and release of stress hormones into the bloodstream. In another exemplary embodiment, no music is played during vibration therapy.

The present invention can be applied to any patient regardless of age, ethnicity, sex, or physical condition. In an exemplary embodiment, the patient is above 50 years of age. In a preferred embodiment, the patient has reduced mobility. In another preferred embodiment, the patient is semi-sedentary. In a further preferred embodiment, the patient is non-ambulatory.

The present invention utilizes multiple benefits of vibration therapy to promote clinical efficacy of another therapeutic modality. Vibration therapy, as a non-invasive modality, assists in neuromodulation and provides axonal regeneration, neurite outgrowth, and/or pain relief.

Vibration therapy provides for sleep improvement, via increased glymphatic system activity, such as glymphatic clearance, neuromodulator delivery, and toxin removal, via decreased harmful microglia activity, via reducing anxiety in a user, via an unlisted mechanism, or via any combination thereof.

Vibration therapy provides for stress reduction, via an enhanced immune response, an increase in expression of antioxidants, muscle relaxation, and reduced inflammation. Vibration therapy also provides improved endocrine response, neurochemical release, and accelerated tissue regeneration.

Vibration therapy provides for stimulation of the parasympathetic nervous system. This may lead to decreased blood pressure, heart rate, and respiration rate. Additionally, a beneficial effect is enhanced transport across the blood-brain barrier (BBB) or another barrier. This transport across the BBB can be bi-directional, that is, into or out of the brain. In an exemplary embodiment, transfer of the therapeutic agent into the brain is increased. In another exemplary embodiment, clearance of waste from the brain is enhanced. In a further exemplary embodiment, both transfer of therapeutic agent into the brain and clearance of waste from the brain are promoted.

Further, low-frequency vibration improves overall cardiovascular health via enhanced lymphatic processes, vagal nerve balancing (which provide anti-inflammatory effects and neurotransmitter release), and/or vasodilation (which results from endothelial cell stimulation and increased nitric oxide production and release).

The present invention combines vibrational treatment with medicinal treatment of a clinical condition, such as a neurological disorder. The clinical effects provided by such combination would be more than the added effects of each treatment when applied alone. In other words, the combinatorial treatment provides for synergistic effects when two treatments are administered within 24 hours or a full wake-sleep cycle of a patient.

Several mechanisms based on multiple benefits of vibration therapy may contribute to the synergy of combining vibration with a therapeutic drug. The mechanisms are not mutually exclusive nor exhaustive. More than one mechanism, illustrated below or not, may be involved at the same time. The specific mechanism(s) involved depends on the neurological disorder to be treated, the therapeutic modalities employed, and the physical conditions of the patient. These mechanisms may also apply to treatment of other clinical conditions, including those not involving the brain.

The first three mechanisms stem from the vasodilation benefit of vibration therapy, and vasodilation may be mediated through increase natural nitric oxide release. Vasodilation increases both vessel diameter and overall volume of blood flow, especially with capillaries, which may have been restricted to single file cell flow or no cell flow states. In addition to enhanced circulatory function, the enlarged vessel diameter and increased overall flow presents more transport surface area and increased volumetric flow of the brain-targeting pharma-therapeutics across the BBB. This creates better opportunity for the drug's transfer into the targeted areas and increased net therapeutic value.

In the meantime, vasodilation promotes waste removal and further improves clinical efficacy of the drug in a clinical condition (such as but not limited to Alzheimer's disease) that involves waste buildup. In an exemplary embodiment, VAT and/or WBV reduces cerebral amyloid load in a patient with Alzheimer's disease, which promotes sleep quality, sleep duration, and overall wellbeing of the patient.

A third mechanism also roots in vasodilation benefit of vibration therapy. As a substitute for cardiovascular exercise, vasodilation serves to increase blood flow, thereby enhance oxygen and nutrient distribution as well as waste removal while lowering blood pressure. This mechanism may be particularly noticeable in semi-sedentary individuals that are unable to participate in other cardiovascular actuation exercises.

A fourth mechanism lies in micro-mechanical tissue stretching. This helps, among other benefits, to stimulate movement within the lymphatic system, which normally requires body movement in order to function properly. This tissue stretching phenomenon has also been shown by numerous studies to accelerate muscle regeneration. The higher amplitude vibration, whole body vibration, has shown positive reduction of osteoporosis and a general increase in bone density.

Vibration therapy is particularly popular in elderly patients, who have found this system to be pleasant and relaxing, thereby encouraging continued use. Reduced anxiety has been observed in nearly all sessions and continued use has led to durable reduction in resting heart rate.

Also provided are apparatus capable of delivering to a user vibration of a frequency and amplitude sufficient to generate tactile appreciation in the user. In a preferred embodiment, the apparatus delivers vibration to a user and places the user into a ventral vagal state. In another preferred embodiment, the apparatus delivers vibration to a user and increases substance transfer across the user's blood-brain barrier.

The apparatus can be of different materials, sizes, shapes, weights, or other configurations. The apparatus can be a supporting surface, a piece of furniture, a wearable, a personal item, a 3D object, another device capable of delivering to a user desired vibration, or any combination thereof. In an exemplary embodiment, the apparatus is a flat platform, for example without limitation, a floor. In another exemplary embodiment, the apparatus is a curved platform, for example without limitation, a lounge chair. In a further exemplary embodiment, the apparatus is a wearable around the neck.

The apparatus delivering vibration to the user can be vibrating on its own or through an outside vibrating source. In an exemplary embodiment, the apparatus provides its own vibrational energy. In another exemplary embodiment, vibrational energy is provided to the apparatus from another source external to the apparatus.

Further provided are combinatorial systems for treating at least one clinical condition in a patient in need thereof. The system includes an apparatus capable of delivering vibration and at least one other therapeutic modality for treating at least one clinical condition. The clinical condition can be a neurological disorder.

The apparatus can be made of different materials, with different sizes, shapes, weights, or other configurations. The apparatus can be vibrating on its own or through another vibrating source.

In an exemplary embodiment, the system includes an apparatus capable of vibrating and at least one therapeutic modality for treating at least one neurological disorder in a patient in need thereof. The platform contains at least one sheet of material with an upper surface and a lower surface, at least one transducer on the lower surface of each sheet for exciting the sheet to vibrate, three or more supports disposed extending from the lower surface of each sheet, and a control unit that controls each transducer. The platform is capable of delivering to a patient vibration of low frequency and sufficient amplitude to generate significant tactile appreciation in the patient.

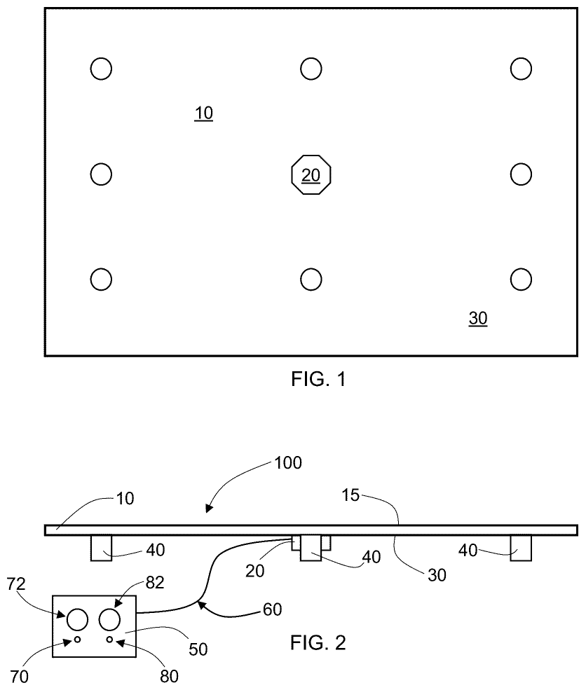

Referring to FIGS. 1 and 2, consistent with certain embodiments in the present invention, a platform 100 for exciting a platform includes at least one exemplary sheet 10, shown in a bottom plan view in FIG. 1. Sheet 10 includes an upper surface 15 and a lower surface 30. In an exemplary embodiment, a transducer 20 is disposed on a lower surface 30 of sheet 10. In an exemplary embodiment, transducer 20 is attached to sheet 10 by an adhesive, by fasteners, or by any mechanism for attachment as is known in the art.

In an exemplary embodiment, transducer 20 excites sheet 10 with acoustic vibrational energy. In another exemplary embodiment, transducer 20 excites sheet 10 with mechanical vibrational energy. In a further exemplary embodiment, transducer 20 excites sheet 10 with a combination of acoustic vibrational energy and mechanical vibrational energy.

In an exemplary embodiment, sheet 10 vibrates horizontally. In another exemplary embodiment, sheet 10 vibrates vertically. In a further exemplary embodiment, sheet 10 alternates between horizontal vibration and vertical vibration.

In an exemplary embodiment, acoustic transducer 20 is a commercially available source for acoustic excitation, for example without limitation, a speaker, a subwoofer, another sound source, or a transducer. In another exemplary embodiment, the system contains a speaker (not shown) in addition to acoustic transducer 20.

In an exemplary embodiment, the vibrational excitation provided by transducer 20 is a sinusoidal vibration. In a further exemplary embodiment, sheet 10 is made from a material that is susceptible to vibrating in response to excitation from transducer 20. In this exemplary embodiment, sheet 10 responds to the vibrational excitation provided by transducer 20 by vibrating in a sinusoidal mode wherein the maximum amplitude of vibration follows a sinusoidal curve. In an exemplary embodiment, sheet 10 is made from a material, for example without limitation, including solid wood, plywood, sheet metal, or any material that can be formed into a sheet and suitable for vibrational excitation as disclosed herein.

In an exemplary embodiment, three or more supports 40 are disposed extending from lower surface 30 of each sheet 10. In an exemplary embodiment, each of the supports 40 comprises a spring, for example a coil spring. In an exemplary embodiment, each of the coil springs 40 is made from a coil of wire and provides a resilient support for sheet 10 via torsional resistance in the wire. Without being held to theory, the spring constants of springs 40 and the distribution of springs 40 along lower surface 30 of sheet 10 are designed to accommodate the combined weight of sheet 10 and any human beings present on sheet 10 at an approximate distribution of about 16 ft² per human. Further, the design parameters for the thickness of sheet 10, the material making up sheet 10, the stiffness of sheet 10, and the size, thickness, spring constant and distribution of springs 40, are selected to allow sheet 10 to freely vibrate at the frequency of excitation of transducer 20, while preventing or minimizing the formation of first and second order harmonic vibrations of sheet 10 below about 20 Hz and above about 240 Hz. Minimizing the formation of the first and second order harmonics of sheet 10 at frequencies outside of the desired range of frequencies prevents undesirable noise, vibration, or potential resonance induced damage to sheet 10.

In an exemplary embodiment, a control unit 50 is disposed in communication with each transducer 20. In an exemplary embodiment, control unit 50 communicates with each transducer 20 via a wire or wires 60. Each of the transducers 20 also requires power to operate, and so can also be connected to a source of power via a wired connection (not shown). In an exemplary embodiment, control unit 50 communicates with each transducer 20 via a wireless connection. In another exemplary embodiment, control unit 50 communicates with each transducer 20 via a wired connection. In other embodiments, control unit 50 communicates with one or more of the transducers 20 with a wired connection and communicates with one or more of the transducers 20 with a wireless connection. In an exemplary embodiment, control unit 50 is configured to send an electrical signal to each transducer 20, and the electrical signal comprises an excitation amplitude and an excitation frequency for transducer 20.

FIG. 2 schematically shows an exemplary control unit 50 including an amplitude control knob 70, an amplitude display 72, a frequency control knob 80, and a frequency display 82. In other embodiments, the actual configuration of control unit 50 can include other types of user interface including slide switches, an alphanumerical keyboard, and the like. Control unit 50 also preferably includes a display for signals received from sheet 10, for example without limitation, from a waveform sensor (not shown) disposed on sheet 10, for the display of the actual frequency and amplitude of vibration of sheet 10.

In an exemplary embodiment, the excitation frequency set on control unit 50 and delivered to transducer 20 is the frequency at which transducer 20 operates. Without being held to theory, in this exemplary embodiment, sheet 10 can vibrate at the set frequency or at a slightly different frequency because of the physical geometry of sheet 10 and other factors. However, in a further exemplary embodiment, control unit 50 is calibrated so that the acoustic excitation frequency set on control unit 50 is the frequency at which sheet 10 vibrates, taking into account any variations with the vibrational frequency of transducer 20. In an exemplary embodiment, transducer 20 excites sheet 10 with vibrational energy so that sheet 10 vibrates in a range from about 20 Hz to about 250 Hz. In an exemplary embodiment, transducer 20 excites sheet 10 with vibrational energy so that sheet 10 vibrates at about 40 Hz. In an exemplary embodiment, 40 Hz has been found to be the most beneficial frequency for vibroacoustic therapy related to several diseases, including, without limitation, Alzheimer's disease, Parkinson's disease, and post-traumatic stress disorder (PTSD).

In an exemplary embodiment, the amplitude set on control unit 50 is simply a setting from minimum to maximum, for example, a low setting of 1 and a high setting of 10. In an exemplary embodiment, the amplitude set on control unit 50 is calibrated to indicate the actual maximum amplitude of vibration for sheet 10 based on the type of material used in sheet 10 and other factors. In an exemplary embodiment, transducer 20 excites sheet 10 with vibrational energy so that sheet 10 vibrates having a maximum amplitude in a range from about 0.001 mm to about 25 mm. In another exemplary embodiment, the maximum amplitude of vibration of sheet 10 can be less than 0.001 mm or greater than 25 mm. In an exemplary embodiment, the maximum amplitude of the vibration of sheet 10 is sufficient to accommodate what are known as camping factors, which include mats or rugs on sheet 10, and further provide sufficient amplitude when sensed through the camping factors to generate significant tactile appreciation for an observer positioned on sheet 10.

In an exemplary embodiment, sheet 10 is provided as a furniture or part of a furniture such as a chair and in direct contact with the patient. In another exemplary embodiment, sheet 10 is indirectly contacting the patient, such as through a piece of furniture not vibrating by itself. In yet another exemplary embodiment, sheet 10 is wearable. In a preferred embodiment, sheet 10 is a floor. The patient can be standing, walking, dancing, sitting, lying, stretching on, or otherwise contacting sheet 10.

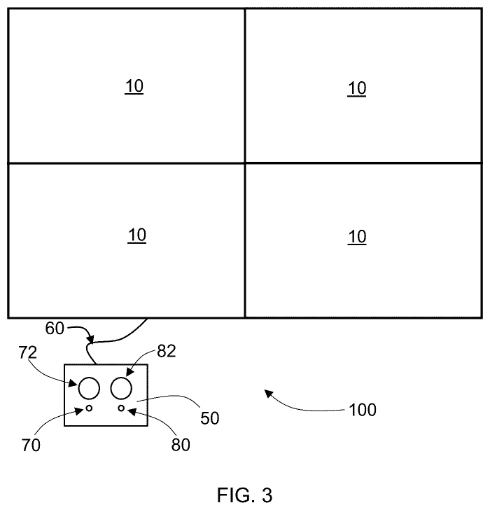

Referring to FIG. 3, in an embodiment, the system 100 comprises two or more of the sheets 10 disposed adjacently when viewed looking at the upper surfaces 15. It should be noted that although the sheets 10 in FIGS. 1 and 3 are illustrated as rectangular that any shape as viewed from above can be used for any individual sheet 10. In an embodiment having more than one sheet 10, a single control unit 50 controls all of the transducers 20. In another embodiment having two or more of the sheets 10 disposed adjacently there are multiple control units 50 each controlling one or more of the transducers 20.

In an embodiment where multiple sheets 10 are combined to form a larger platform, adjacent edges of the sheets 10 are lined up. In an embodiment the lined up adjacent edges are left free from one another, with a small gap left therebetween. In an embodiment the lined up adjacent edges are not attached but do touch one another. In another embodiment the adjacent edges are physically attached to one another, for example without limitation, by an adhesive, an adhesively applied flexible sheet, or otherwise. Without being held to theory, in an embodiment the physical attachment of adjacent edges of multiple sheets 10 does not affect the frequency, amplitude, or modes of vibration of the individual sheets 10.

In a preferred embodiment, speaker is an audio speaker. Speaker can alternately be headphones, earbuds, or any device that is capable of playing music to user. Research has shown that different sound frequencies can have distinct physiological effects on the body of a user. In accordance with an exemplary embodiment, the music played through speakers includes low-frequency sounds, which have been found to place a listener in a ventral vagal state, which is one state of the nervous system's parasympathetic response, which promotes relaxation and a lack of anxiety in a listener. Being in a ventral vagal state has been found to lead to better digestion, improved immune response, increased circulation, lower blood pressure, decreased muscle tension, and enhanced relaxation. In a further exemplary embodiment, the music played through the speaker is any music that places a listener in a ventral vagal state, such as classical music or the like.

The methods, apparatus, and systems provided herein can be used by individuals or by professionals administering therapy to individuals.

Numerous modifications to the present invention will be apparent to those skilled in the art in view of the foregoing description. It is not desired to limit the invention to the exact construction and operation shown and described, and accordingly, all suitable modifications and equivalents may be resorted to, falling within the scope of the invention. Accordingly, this description is to be construed as illustrative only of the principles of the invention and is presented for the purpose of enabling those skilled in the art to make and use the invention and to teach the best mode of carrying out same. The exclusive rights to all modifications which come within the scope of the appended claims are reserved. All patents, patent publications and applications, and other references cited herein are incorporated by reference herein in their entirety.

1. A method for treating a neurological disorder in a patient in need thereof, the method comprising:
subjecting the patient to a vibroacoustic therapy for a period of time; and
administering at least one other therapeutic modality to the patient, wherein the at least one other therapeutic modality comprises a therapeutic agent clinically indicated for treatment of the neurological disorder, wherein the at least one other therapeutic modality is not vibrational or light-based, and wherein the administration of the at least one therapeutic modality occurs before, during, or after the vibroacoustic therapy.

2. The method of claim 1, wherein the vibroacoustic therapy is of an appropriate frequency, amplitude, and duration such that the vibroacoustic therapy promotes vasodilation and increases transfer across the patient's blood-brain barrier.

3. (canceled)

4. The method of claim 1, wherein the vibroacoustic therapy is generated and maintained by acoustic means, mechanical means, or a combination thereof.

5. The method of claim 1, wherein the vibration of the vibroacoustic therapy comprises vertical vibration, horizontal vibration, or any combination thereof.

6. The method of claim 2, wherein the frequency of the vibroacoustic therapy is within a range between 20 Hz and 250 Hz.

7. (canceled)

8. The method of claim 1, wherein a maximum amplitude of the vibroacoustic therapy is within a range from 0.001 mm to 25 mm.

9. The method of claim 1, wherein the neurological disorder is Alzheimer's Disease, other dementias, Parkinson's Disease, Huntington's disease, sclerosis, depression, Post-Traumatic Stress Disorder, or any combination thereof.

10. The method of claim 1, wherein the at least one other therapeutic modality is administered within a time window ranging from 24 hours before the vibroacoustic therapy to 24 hours after the vibroacoustic therapy.

11. (canceled)

12. The method of claim 1, wherein the therapeutic agent is aducanumab, donanemab, lecanemab, carbidopa, levodopa, rotigotine, paroxetine, venlafaxine, haloperidol, tetrabenazine, amantadine, or any combination thereof.

13. The method of claim 1, wherein the therapeutic agent is administered to the patient via ingestion, inhalation, intracerebral injection, intravenous injection, subcutaneous injection, intramuscular injection, intrathecal administration, intraperitoneal administration, transdermal administration, sublingual administration, intranasal administration, or any combination thereof.

14. The method of claim 1, wherein the patient is in contact with an apparatus configured to deliver to the patient the vibroacoustic therapy that increases transfer across the patient's blood-brain barrier.

15. (canceled)

16. A system for treating a neurological disorder in a patient in need thereof, the system comprising:
an apparatus configured to deliver to the patient a vibroacoustic therapy of an appropriate frequency, amplitude, and duration to increase transfer across the patient's blood-brain barrier; and
at least one other therapeutic modality for treating the neurological disorder, wherein the at least one other therapeutic modality comprises a therapeutic agent clinically indicated for treatment of the neurological disorder, and wherein the at least one other therapeutic modality is not vibrational or light-based.

17. The system of claim 16, further comprising a speaker configured to play music that promotes a ventral vagal state in the patient.

18. (canceled)

19. (canceled)

20. (canceled)

21. The method of claim 2, wherein the vibroacoustic therapy is configured to increase nitric oxide release from endothelial cells and bi-directional transfer across the patient's blood-brain barrier so that an effect on treatment of the neurological disorder that is greater than that of each treatment alone is obtained.

22. The method of claim 14, wherein the apparatus comprises a vibrating platform.

23. The system of claim 16, wherein the apparatus comprises a vibrating platform.

24. The system of claim 23, wherein the vibrating platform comprises a sheet of material with an upper surface and a lower surface, and at least one transducer disposed on the lower surface for exciting the sheet to vibrate.
